(19) United States
(12) Patent Application Publication
(10) Pub. No.: US 2011/0144315 A1
(43) Pub. Date: Jun. 16, 2011

(54) CONTROLLED SYNTHESIS OF POLYGLUTAMIC ACID

(75) Inventors: Hai Wang, San Diego, CA (US); Wendy Dianne Taylor, San Diego, CA (US)

(73) Assignee: NITTO DENKO CORPORATION, Osaka (JP)

(21) Appl. No.: 12/967,352

(22) Filed: Dec. 14, 2010

Related U.S. Application Data
(60) Provisional application No. 61/287,129, filed on Dec. 16, 2009.

(51) Int. Cl. C07K 1/12

(52) U.S. Cl. 530/407

(57) ABSTRACT

Disclosed herein are processes for obtaining polyglutamic acid. The processes disclosed herein are controlled processes for obtaining polyglutamic acid with a desired weight average molecular weight.

### RELATED APPLICATIONS

This application claims the benefit of U.S. Provisional Application No. 61/287,129, filed Dec. 16, 2009, which is incorporated by reference herein in its entirety, including any drawings.

### BACKGROUND

1. Field

Generally disclosed herein are processes for obtaining polyglutamic acid. More specifically, disclosed herein are controlled processes for obtaining polyglutamic acid with a desired weight average molecular weight.

2. Description

Polyglutamic acid is commercially available. However, the price is expensive, approximately $350-$500 per gram (Sigma Aldrich Chemical Company). The high price is associated with the difficulty of synthesizing polyglutamic acid with a particular molecular weight. Polyglutamic acid is often obtained by using an initiator to initiate the polymerization of N-carboxyanhydride. The polymerization reaction is terminated when a particular molecular weight is believed to be achieved. However, it is difficult to predict when to terminate the reaction in order to obtain a particular molecular weight of polyglutamic acid. Furthermore, it is difficult to produce polyglutamic acid with a low polydispersity index. It is also difficult to produce polyglutamic acid on a large scale.

### SUMMARY

Some embodiments disclosed herein relate to a process for preparing polyglutamic acid that can be used to obtain polyglutamic acid with a desired weight average molecular weight within a narrow range of kiloDaltons (kDa). In addition to obtaining a desired weight average molecular weight of polyglutamic acid with relative accuracy, in some embodiments, the process is less expensive than the commercial methods currently available. Additionally, in some embodiments, the process can be used to obtain polyglutamic acid on a 10 g to 1000 g scale level.

Some embodiments can include obtaining a starting polyglutamic acid having a first weight average molecular weight equal to or greater than 80 kDa; selecting a target second weight average molecular weight of polyglutamic acid that is less than 80 kDa; selecting hydrolyzing conditions that are effective to reduce the first weight average molecular weight of the starting polyglutamic acid to the selected target second weight average molecular weight of polyglutamic acid; and hydrolyzing the starting polyglutamic acid under the hydrolyzing conditions to thereby obtain a product polyglutamic acid, wherein the product polyglutamic acid has a weight average molecular weight that is within about ±10 kDa of the selected target second weight average molecular weight.

Some embodiments can include obtaining a starting polyglutamic acid having a first weight average molecular weight equal to or greater than 185 kDa; selecting a target second weight average molecular weight of polyglutamic acid that is less than 185 kDa; selecting hydrolyzing conditions that are effective to reduce the first weight average molecular weight of the starting polyglutamic acid to the selected target second weight average molecular weight of polyglutamic acid; and hydrolyzing the starting polyglutamic acid under the hydrolyzing conditions to thereby obtain a product polyglutamic acid, wherein the product polyglutamic acid has a weight average molecular weight that is within about ±10 kDa of the selected target second weight average molecular weight.

These and other embodiments are described in greater detail below.

### BRIEF DESCRIPTION OF THE DRAWINGS

FIG. 1 shows a plot that illustrates the weight average molecular weight of polyglutamic acid obtained by hydrolyzing a polyglutamic benzyl ester with a starting weight average molecular weight of 191 kDa under hydrolysis conditions that include HBr-AcOH for several hours at 30° C.

FIG. 2 shows a plot that illustrates the weight average molecular weight of polyglutamic acid obtained by hydrolyzing two polyglutamic benzyl ester samples with starting weight average molecular weights of 130 kDa under hydrolyzing conditions that include HBr-AcOH for several hours at 30° C.

### DETAILED DESCRIPTION

### I. Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “polyglutamic acid” or “PGA” is used herein according to its ordinary meaning as understood by those skilled in the art. Those skilled in the art understand that at certain pH levels (for example pH>7) the hydrogens attached to pendant carboxylic acid groups of polyglutamic acid can be replaced with an appropriate cation, such as sodium. Thus, polyglutamic acid includes polymers comprised of glutamic acid monomer units wherein the pendant carboxylic acid is either protonated or deprotonated. Deprotonated glutamic acid monomer units of polyglutamic acid include glutamate salts such as sodium salts, potassium salts, lithium salts, calcium salts, magnesium salts, and ammonium salts (such as tetrabutylammonium (TBA), tetrapropylammonium (TPA), hexadecyltrimethylammonium, dodecyltriethylammonium, tetramethylammonium, tetraethylammonium, and tris(hydroxymethyl)aminomethane salts), and combinations thereof.

In some embodiments, the terminal hydrogens of the carboxylic acid groups can be replaced with suitable protecting groups. Thus, polyglutamic acid includes unprotected polyglutamic acid and protected polyglutamic acid. Suitable protecting groups are known to those skilled in the art. Ester protecting groups include, but are not limited to, C₁-C₁₄ alkyl esters, C₆-C₁₀ aryl esters, and C₇-C₁₄ aralkyl esters. Exemplary ester protecting groups for polyglutamic acid include, but are not limited to, phenyl ester, benzyl ester, alkyl esters (such as methyl ester, ethyl ester, propyl ester, isopropyl ester, butyl ester, t-butyl ester, and heptyl ester), and any other ester protecting group known in the art. See, e.g., Wuts and Greene, Greene's Protective Groups in Organic Synthesis; John Wiley and Sons, 2007. In some embodiments, the protecting group can be a benzyl ester, such as benzylic ester.

Thus, the term “polyglutamic acid” or “PGA” is a general term that includes variants such as polyglutamate and polyglutamic acid in which the hydrogens of the carboxylic acid groups have been replaced with counterions and/or suitable protecting groups. Polyglutamic acid includes both poly-alpha-glutamic acid and poly-gamma-glutamic acid. For example, the term “polyglutamic acid” includes poly-alpha-glutamic acid-gamma-(benzyl)ester and poly-alpha-glutamic acid-gamma-(t-butyl)ester. Polyglutamic acid includes polymers wherein 75% or more of the monomer units are glutamic acid monomer units.

As used herein, the terms “hydrolysis” and “hydrolyzing” refer to the cleavage of protecting groups from the polyglutamic acid and/or the cleavage of amide backbone bonds in the polyglutamic acid.

As used herein, the term “hydrolyzing condition” refers to chemical reaction parameters that result in hydrolysis. Exemplary hydrolyzing condition parameters include, but are not limited to, time, temperature, solvent, and hydrolyzing reagents. Exemplary hydrolyzing reagents include, but are not limited to, acid reagents, basic reagents, and/or enzymatic reagents. Hydrolyzing conditions are generally known in the art. See, e.g., Smith and March, March's Advanced Organic Chemistry, John Wiley & Sons, 2007, pages 1400-1411.

As used herein, the term “weight average molecular weight” or “M_{w}” can be used to describe the molecular weight of a polymer. The weight average molecular weight is the sum of the products of the molar mass of each fraction multiplied by its weight fraction. See, e.g., Young, Introduction to Polymers, Chapman and Hall, 1981, page 8; and Stevens, Polymer Chemistry: An Introduction, Oxford University Press, pages 35-37.

As used herein, the term “number average molecular weight” or “ M̅ₙ” can be used to describe the molecular weight of a polymer. The number average molecular weight is the sum of the products of the molar mass of each fraction multiplied by its mole fraction. See, e.g., Young, Introduction to Polymers, Chapman and Hall, 1981, page 5; and Stevens, Polymer Chemistry: An Introduction, Oxford University Press, pages 35-37.

As used herein, the term “polydispersity index” refers to the ratio of the weight average molecular weight to the number average molecular weight. The polydispersity index can be expressed mathematically as M̅_{w}/ M̅ₙ.

It is understood that, when a range of numbers is provided herein, the range is intended to include each integer within the provided range of numbers. For example, it is understood that reference to polymers having a molecular weight in the range of 20-25 kDa is a description of various polymers with molecular weights of 20 kDa, 21 kDa, 22 kDa, 23 kDa, 24 kDa, and 25 kDa. Similarly, it is understood that reference to temperatures in the range of 20-40° C. is a description of various temperatures of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., and 40° C.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z each double bond may independently be E or Z a mixture thereof. Likewise, all tautomeric forms are also intended to be included.

### II. Polyglutamic Acid

Disclosed herein are processes for preparing polyglutamic acid. Some embodiments disclosed herein relate to a process for preparing polyglutamic acid that can include obtaining a starting polyglutamic acid having a first weight average molecular weight equal to or greater than 185 kDa; selecting a target second weight average molecular weight of polyglutamic acid that is less than 185 kDa; selecting hydrolyzing conditions that are effective to reduce the first weight average molecular weight of the starting polyglutamic acid to the selected target second weight average molecular weight of polyglutamic acid; and hydrolyzing the starting polyglutamic acid under the hydrolyzing conditions to thereby obtain a product polyglutamic acid, wherein the product polyglutamic acid has a weight average molecular weight that is within about ±10 kDa of the selected target second weight average molecular weight.

In some embodiments, the process for preparing polyglutamic acid can include the step of selecting a starting polyglutamic acid having a first weight average molecular weight between 50 kDa to 500 kDa. The process for preparing polyglutamic acid can include the step of selecting a starting polyglutamic acid having a first weight average molecular weight of up to 100,000 kDa.

The process for preparing polyglutamic acid can include the step of selecting a target second molecular weight polyglutamic acid that has a molecular weight less than that of the starting polyglutamic acid.

The process for preparing polyglutamic acid can include the step of selecting acidic, basic, or enzymatic hydrolysis conditions that are effective for reducing the weight average molecular weight of the starting polyglutamic acid to the target second weight average molecular weight.

The process for preparing polyglutamic acid can produce a product polyglutamic acid with a lower weight average molecular weight that is within about ±5 to ±50 kDa of the selected target second weight average molecular weight. The process for preparing polyglutamic acid can produce a product polyglutamic acid with a lower weight average molecular weight that is within ±1% to ±10% of the selected target second weight average molecular weight.

A. Starting Polyglutamic Acid

The starting polyglutamic acid can be obtained from various sources. For example, the starting polyglutamic acid can be obtained from a commercial source such as Sigma-Aldrich Chemical Co. Alternatively, the starting polyglutamic acid can be synthesized. Suitable methods for the synthesizing starting polyglutamic acid are known to those skilled in the art. One method for synthesizing the starting polyglutamic acid is by reacting a glutamic ester monomer with a suitable initiator. An example of a suitable reaction between a glutamic ester monomer and an initiator is illustrated in Scheme 1A.

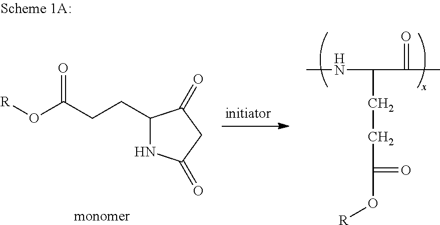

wherein R is an ester protecting group. Any ester protecting group known in the art or previously mentioned herein can be used. In some embodiments R is C₁-C₁₄ alkyl, C₆-C₁₀ aryl, or C₇-C₁₄ aralkyl. In some embodiments, R is benzyl, phenyl, t-butyl, isopropyl, ethyl, or methyl.

For example, a benzyl ester glutamic acid N-carboxyanhydride can be reacted with an amine initiator to produce a polyglutamic acid benzyl ester polymer, as illustrated in Scheme 1B. The amine initiator can be triethyl amine (TEA). The reaction can be performed in dioxane at room temperature.

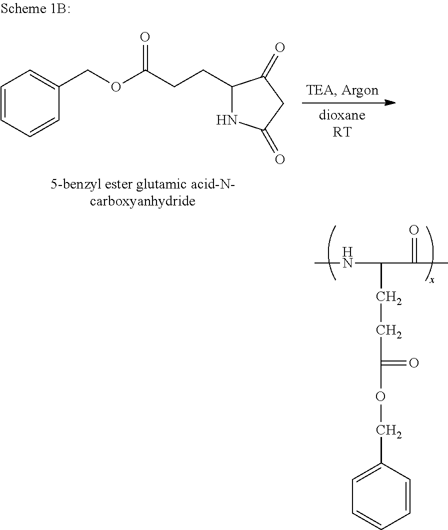

i. Starting Polyglutamic Acid Molecular Weight

The starting polyglutamic acid has a higher weight average molecular weight as compared to the product polyglutamic acid that results from the hydrolysis. In some embodiments, the starting polyglutamic acid has a first weight average molecular weight equal to or greater than 80 kDa. In some embodiments, the starting polyglutamic acid has a first weight average molecular weight equal to or greater than 100 kDa. In some embodiments, the starting polyglutamic acid has a first weight average molecular weight equal to or greater than 130 kDa. In some embodiments, the starting polyglutamic acid has a first weight average molecular weight equal to or greater than 150 kDa. In some embodiments, the starting polyglutamic acid has a first weight average molecular weight equal to or greater than 170 kDa. In some embodiments, the starting polyglutamic acid has a first weight average molecular weight equal to or greater than 185 kDa. Methods for determining the weight average molecular weight of the starting polyglutamic acid are known to those skilled in the art. Various methods include, but are not limited to, size exclusion chromatography-high pressure liquid chromatography (SEC-HPLC) using appropriate molecular weight detection technology (e.g., light scattering), small angle neutron scattering (SANS), X-ray scattering, and sedimentation velocity. SEC-HPLC may also be referred to as gel permeation chromatography (GPC). If two or more methods for determining the weight average molecular weight of the polyglutamic acid polymer produce different molecular weight values, then the weight average molecular weight value obtained by SEC-HPLC is preferred. In some embodiments, the starting polyglutamic acid can have a first weight average molecular weight equal to or greater than 190 kDa. In other embodiments, the starting polyglutamic acid can have a first weight average molecular weight equal to or greater than 200 kDa. In still other embodiments, the starting polyglutamic acid can have a first weight average molecular weight equal to or greater than 220 kDa. In yet still other embodiments, the starting polyglutamic acid can have a first weight average molecular weight equal to or greater than 230 kDa. In some embodiments, the starting polyglutamic acid can have a first weight average molecular weight equal to or greater than 240 kDa.

In some embodiments, the starting polyglutamic acid has a first weight average molecular weight in the range of about 50 kDa to about 500 kDa. In some embodiments, the starting polyglutamic acid has a first weight average molecular weight in the range of about 80 kDa to about 300 kDa. In some embodiments, the starting polyglutamic acid has a first weight average molecular weight in the range of about 80 kDa to about 130 kDa. In some embodiments, the starting polyglutamic acid has a first weight average molecular weight in the range of about 130 kDa to about 270 kDa. In some embodiments, the starting polyglutamic acid has a first weight average molecular weight equal to or greater than 80 kDa. In some embodiments, the starting polyglutamic acid has a first weight average molecular weight equal to or greater than 40 kDa.

ii. Initiator for Polymerization

In general, the polymerization initiator shown in Scheme 1A is a nucleophile. In addition, the polymerization initiator preferably possesses physical properties which enable the initiator to be separated from the product polymer or otherwise eliminated from the reaction mixture upon the completion of the polymerization reaction. Exemplary initiators include benzylamine, n-hexylamine, diethylamine, triethylamine, sodium methoxide, sodium N-benzylcarbamate, sodium hydroxide, sodium borohydride, sodium ethoxide, sodium propoxide, potassium methoxide, potassium ethoxide, potassium propoxide, potassium tert-butoxide, diisopropylethylamine, 1,8-diazabicyclo[5,4,0]undec-7-ene (DBU), 4-dimethylaminopyridine (DMAP), glutamic acid dimethyl ester, and glutamic acid-gamma-tert-butyl ester, or any anionic ring opening initiator known in the art.

B. Target Molecular Weight

In some embodiments, the selected target second weight average molecular weight equal to or less than 40 kDa. In some embodiments, the selected target second weight average molecular weight can be in the range of about 40 kDa to about 12 kDa. In other embodiments, the selected target second weight average molecular weight can be in the range of about 30 kDa to about 15 kDa. In still other embodiments, the selected target second weight average molecular weight can be in the range of about 25 kDa to about 20 kDa. There are various reasons for selecting a certain selected target weight average molecular weight. A non-limiting list of reasons include increased solubility of the polyglutamic acid with the selected target weight average molecular weight, decreasing and/or preventing secretion of the polyglutamic from the body (for example, from the kidney) and decreasing the immunoresponse of the body to the polyglutamic acid.

In addition, properties such as in vivo degradation time, blood circulation time, biocompatibility, toxicity, antigenic potential, immunogenic stimulation, biological stability, hydrolytic stability, enzymatic stability, solubility, permeability, swelling, glass transition temperature, melting temperature, decomposition temperature, modulus, tensile strength, elasticity, and diffusivity transport can depend on the molecular weight of the selected target polyglutamic acid polymer.

In some embodiments, the selected target second weight average molecular weight can be in the range of about 100 kDa to about 1 kDa. In some embodiments, the selected target second weight average molecular weight can be in the range of about 100-80 kDa, 90-70 kDa, 80-60 kDa, 70-50 kDa, 60-40 kDa, 50-30 kDa, 40-20 kDa, 30-10 kDa, or 20-1 kDa. In some embodiments, the selected target second weight average molecular weight can be in the range of about 45-35 kDa, 40-35 kDa, 35-30 kDa, 30-25 kDa, 25-20 kDa, 22-17 kDa, 20-15 kDa, 15-10 kDa, 10-5 kDa, or 5-2 kDa.

In some embodiments, the selected target second weight average molecular weight is 30 kDa±10%, 29 kDa±10%, 28 kDa±10%, 27 kDa±10%, 26 kDa±10%, 25 kDa±10%, 24 kDa±10%, 23 kDa±10%, 22 kDa±10%, 21 kDa±10%, 20 kDa±10%, 19 kDa±10%, 18 kDa±10%, 17 kDa±10%, 16 kDa±10%, 15 kDa±10%, 14 kDa±10%, 13 kDa±10%, 12 kDa±10%, 11 kDa±10%, or 10 kDa±10%.

In some embodiments, the selected target second weight average molecular weight is 30 kDa±5%, 29 kDa±5%, 28 kDa±5%, 27 kDa±5%, 26 kDa±5%, 25 kDa±5%, 24 kDa±5%, 23 kDa±5%, 22 kDa±5%, 21 kDa±5%, 20 kDa±5%, 19 kDa±5%, 18 kDa±5%, 17 kDa±5%, 16 kDa±5%, 15 kDa±5%, 14 kDa±5%, 13 kDa±5%, 12 kDa±5%, 11 kDa±5%, or 10 kDa±5%.

In some embodiments, the selected target second weight average molecular weight is about 30 kDa, 29 kDa, 28 kDa, 27 kDa, 26 kDa, 25 kDa, 24 kDa, 23 kDa, 22 kDa, 21 kDa, 20 kDa, 19 kDa, 18 kDa, 17 kDa, 16 kDa, 15 kDa, 14 kDa, 13 kDa, 12 kDa, 11 kDa, or 10 kDa.

In some embodiments, the selected target second weight average molecular weight is less than 40 kDa. In some embodiments, the selected target second weight average molecular weight is less than 30 kDa. In some embodiments, the selected target second weight average molecular weight is less than 20 kDa.

C. Hydrolyzing Conditions

The starting polyglutamic acid can hydrolyzed to produce a product polyglutamic acid. The weight average molecular weight of the product polyglutamic acid can be less than the weight average molecular weight of the starting polyglutamic acid. One method for hydrolyzing the starting polyglutamic acid is by subjecting the starting polyglutamic acid to hydrolyzing conditions, as illustrated in Scheme 2.

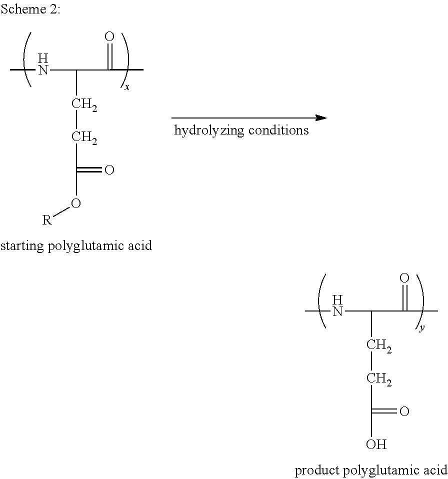

where R represents an ester protecting group; x and y represent integers; and x is greater than y (i.e., x>y).

In some embodiments the product polyglutamic acid can be protonated or deprotonated. In some embodiments, the product polyglutamic acid contains glutamate salt residues such as sodium salts, potassium salts, lithium salts, calcium salts, magnesium salts, and ammonium salts (such as tetrabutylammonium (TBA), tetrapropylammonium(TPA), hexadecyltrimethylammonium, dodecyltriethylammonium, tetramethylammonium, tetraethylammonium, and tris(hydroxymethyl)aminomethane salts).

In some embodiments, the hydrolyzing conditions cleave the protecting groups from the starting polyglutamic acid. In some embodiments the hydrolyzing conditions cleave the backbone amide bonds in the starting polyglutamic acid. In some embodiments the hydrolyzing conditions cleave both protecting groups and backbone amide bonds in the starting polyglutamic acid.

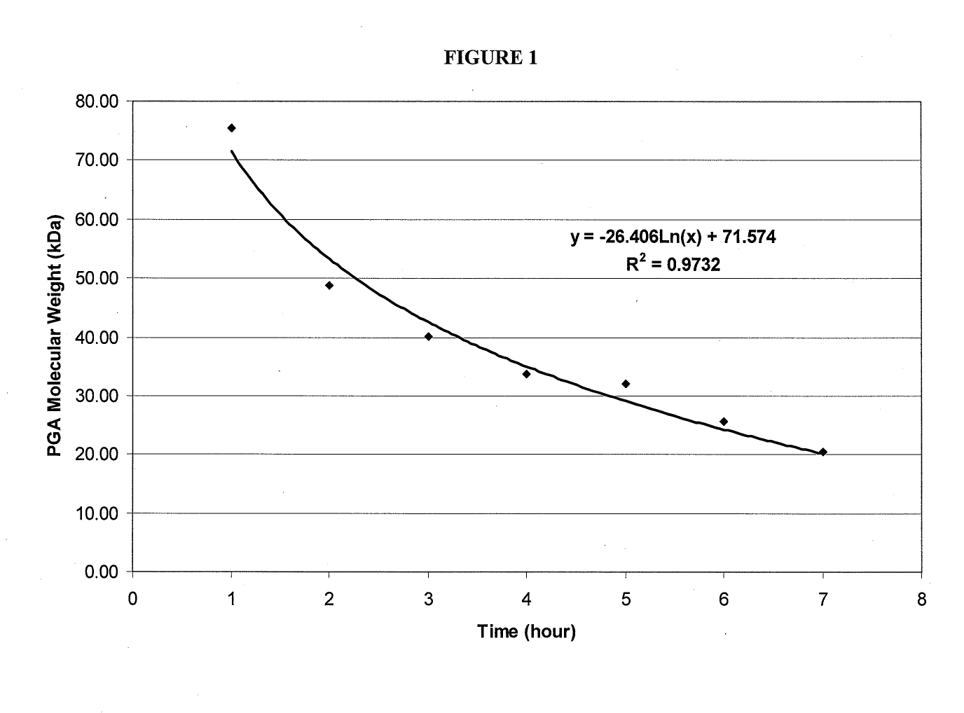

Various conditions can be used to hydrolyze the starting polyglutamic acid. Suitable methods for selecting appropriate hydrolyzing conditions are known to those skilled in the art. In some embodiments, the hydrolyzing conditions include the use of an acid. Suitable acids are known to those skilled in the art. In some embodiments, the acid can be a protic acid. For example, the acid can be hydrobromic acid, hydrochloric acid and sulfuric acid. If needed and/or desired, the acid can be diluted in a protic solvent, for example, water, acetic acid and/or dichloroacetic acid. In an embodiment, the acid can be HBr-acetic acid (HBr-AcOH). In some embodiments, the acid can have a percent composition by mass in the range of about 20% to about 60%. In other embodiments, the acid can have a percent composition by mass in the range of about 30% to about 40%. In still other embodiments, the acid can have a percent composition by mass of approximately 33%. In some embodiments, the hydrolyzing conditions can be selected based on a plot generated from experiments in which polyglutamic acid with a weight average molecular weight greater than the selected target second weight average molecular weight has been subjected to various hydrolyzing conditions. An example of such a plot is shown in FIG. 1. In some embodiments, the polyglutamic acid used to create the plot can be obtained commercially. In other embodiments, the polyglutamic acid used to create the plot can be synthesized, for example using a procedure as described herein.

i. Selecting Hydrolyzing Conditions

Hydrolyzing conditions include reaction parameters that result in cleavage of protecting groups and/or the cleavage of amide backbone bonds in a polyglutamic acid polymer. Exemplary hydrolyzing condition parameters include, but are not limited to, hydrolyzing reagents, temperature, time, solvent, and concentration. Various hydrolyzing condition parameters can be adjusted to produce a product polyglutamic acid polymer having a target weight average molecular weight. Therefore, selecting appropriate hydrolyzing condition parameters will produce a product polyglutamic acid with a target weight average molecular weight.

For example, higher hydrolyzing temperatures generally increase the rate and/or amount of hydrolysis of the polyglutamic acid. Therefore, selecting a higher hydrolyzing temperature will tend to produce a product polyglutamic acid with a lower weight average molecular weight than if a lower temperature were selected.

Furthermore, increasing the amount of time in which a polyglutamic acid polymer is subjected to hydrolyzing conditions will generally increase the amount of hydrolysis of the polyglutamic acid. Therefore, selecting a longer hydrolyzing time will tend to produce a product polyglutamic acid with a lower weight average molecular weight than if a shorter time were selected.

Similarly, utilizing a stronger hydrolyzing reagent will generally increase the rate and/or amount of hydrolysis of the polyglutamic acid. Therefore, selecting a stronger hydrolyzing reagent will tend to produce a product polyglutamic acid polymer with a lower weight average molecular weight than if a weaker hydrolyzing reagent were selected. For example, a stronger acidic reagent will tend to produce a lower weight average molecular weight product polyglutamic acid than a weaker acidic reagent. Likewise, a stronger basic reagent will tend to produce a lower weight average molecular weight product polyglutamic acid than a weaker basic reagent.

ii. Hydrolyzing Reagents

The hydrolyzing conditions illustrated in Scheme 2 include acidic, basic, and enzymatic conditions. Various reagents can be utilized to realize the selected hydrolyzing conditions to produce a desired product polyglutamic acid having a target weight average molecular weight from the starting polyglutamic acid.

In some embodiments acidic hydrolysis conditions are utilized. Acidic conditions can be produced in a solution with a pH of between 6 and 1. Reagents that can produce acidic hydrolyzing conditions include, but are not limited to, HCl, HBr, HF, HClO₄, HClO₃, HClO₂, HClO, H₂SO₄, HNO₃, H₃PO₄, acetic acid, HCO₂H, Cl₂CHCO₂H, cationic exchange resins, or any combination thereof.

For example, the hydrolyzing conditions illustrated in Scheme 2 include a mixture of HBr and acetic acid in Cl₂CHCO₂H. In some embodiments, the hydrolyzing conditions include a mixture of HCl and acetic acid in Cl₂CHCO₂H.

In some embodiments basic hydrolysis conditions are utilized. Basic conditions can be produced in a solution with a pH of between 8 and 14. Reagents that can produce basic hydrolyzing conditions include, but are not limited to, alkali metal hydroxides (such as NaOH, KOH, LiOH, Ba(OH)₂, Cu(OH)₂), t-BuOK, NaH, anionic exchange resins, or any combination thereof.

In some embodiments enzymatically catalyzed hydrolyzing conditions are utilized. Enzymes that can be used include, but are not limited to, esterases (such as pig liver esterase, and esterase from Bacillus subtilis), anhydrases (such as carbonic anhydrase), lipases (such as porcine pancreatic lipase, thermitase, and lipases from Rhizopus niveus, Aspergillus niger, Candida antarcitica, and Mucor javanicus), and any other enzyme known to hydrolyze chemical bonds.

iii. Temperature

In addition to the use of one or more hydrolyzing reagents, the hydrolyzing conditions can comprise subjecting the starting polyglutamic polymer to elevated temperatures. In some embodiments, the starting polyglutamic acid polymer can be subjected to a temperature greater than or equal to about 60° C. In other embodiments, the starting polyglutamic acid polymer can be subjected to a temperature greater than or equal to about 50° C. In still other embodiments, the starting polyglutamic acid polymer can be subjected to a temperature greater than or equal to about 40° C. In some embodiments, the starting polyglutamic acid polymer can be subjected to a temperature in the range of about 40° C. to about 60° C. Alternatively, the starting polyglutamic acid polymer can be subjected to the hydrolyzing conditions at room temperature, approximately 25° C. In some embodiments the hydrolyzing conditions can comprise a temperature in the range of −40° C. to 300° C.

iv. Time

The starting polyglutamic acid can be subjected to hydrolyzing conditions for various amounts of time. In some embodiments, the polyglutamic acid polymer is subjected to hydrolyzing conditions for a period of time in the range of 1 to 120 minutes. In some embodiments, the polyglutamic acid polymer is subjected to hydrolyzing conditions for a period of time in the range of 1 to 24 hours. In some embodiments, the polyglutamic acid polymer is subjected to hydrolyzing conditions for a period of time in the range of about 1 day to about 3 days. In some embodiments, the polyglutamic acid polymer is subjected to hydrolyzing conditions for greater than 3 days. It is understood that, when a range of time is disclosed herein, the range includes each integer and decimal fraction thereof contained within the provided time range. For example, a time range of 1-2 hours is a description of a time of 1.0, 1.1, 1.2, 1.3, 1.4, 1.5, 1.6, 1.7, 1.8, 1.9, and 2.0 hours.

In some embodiments, the hydrolyzing conditions can comprise subjecting the starting polyglutamic acid polymer to a first temperature for a first period of time and a second temperature for a second period of time. As an example, the starting polyglutamic acid polymer can be subjected to a first temperature as described above for a first period of time and a second temperature as described above for a second period of time, wherein the first temperature and the second temperature are different. In some embodiments, the second temperature can be less than the first temperature. In other embodiments, the second temperature can be greater than the first temperature. In still other embodiments, the first and second temperatures can be approximately the same. For example, the starting polyglutamic acid polymer can be subjected to a first temperature in the range of about 40° C. to about 60° C. for a first period of time and room temperature for a second period of time.

The time the starting polyglutamic acid is subjected to the first temperature and the second temperature can vary. For example, the time period of the first temperature can be different from the time period of the second temperature. As an example, the starting polyglutamic acid polymer can be subjected to the first temperature for a first period of time that can be greater or less than a second period of time associated with the second temperature. Alternatively, the time period of the first and second temperatures can be approximately equal. In some embodiments, the first period of time can be equal to or less than 3 hours. In other embodiments, the first time period can be equal to or less than 2 hours. In other still embodiments, the first time period can be equal to or less than 1 hour. In some embodiments, the second time period can be equal to or greater than 1 hour. In other embodiments, the second time period can be equal to or greater than 2 hours. In still other embodiments, the second time period can be equal to or greater than 3 hours. In yet still other embodiments, the second time period can be equal to or greater than 4 hours.

In some embodiments, the first time period is in the range of 1 minute to 120 minutes. In some embodiments, the first time period is in the range of 1 hour to 24 hours. In some embodiments, the first time period is in the range of 1 day to 3 days. In some embodiments, the first time period is for more than 3 days.

In some embodiments, the second time period is in the range of 1 minute to 120 minutes. In some embodiments, the second time period is in the range of 1 hour to 24 hours. In some embodiments, the second time period is in the range of 1 day to 3 days. In some embodiments, the second time period is for more than 3 days.

The total time the starting polyglutamic acid polymer can be subjected to the selected hydrolyzing conditions can vary. In some embodiments, the starting polyglutamic acid can be hydrolyzed under the selected hydrolyzing conditions for at least a total of 2 hours. In other embodiments, the starting polyglutamic acid can be hydrolyzed under the selected hydrolyzing conditions for at least a total of 2.5 hours. In still other embodiments, the starting polyglutamic acid can be hydrolyzed under the selected hydrolyzing conditions for at least a total of 3 hours. In yet still other embodiments, the starting polyglutamic acid can be hydrolyzed under the selected hydrolyzing conditions for at least a total of 4 hours. In some embodiments, the starting polyglutamic acid can be hydrolyzed under the selected hydrolyzing conditions for at least a total of 5 hours, at least a total of 6 hours or at least a total of 7 hours. In other embodiments, the starting polyglutamic acid can be hydrolyzed under the selected hydrolyzing conditions for less than a total of 8 hours.

v. Scale

Various amounts of the starting polyglutamic acid polymer can be subjected to the hydrolyzing conditions described herein. The processes described herein are particularly useful for large scale production. In some embodiments, the amount of starting polyglutamic acid polymer subjected to hydrolyzing conditions in any particular batch is in the range of 10 grams to 100 grams. In some embodiments, the amount of starting polyglutamic acid polymer subjected to hydrolyzing conditions in any particular batch is in the range of 100 grams to 1,000 grams. In some embodiments, the amount of starting polyglutamic acid polymer subjected to hydrolyzing conditions in any particular batch is in the range of 1 kilogram to 10 kilograms.

vi. Solvents

Any solvent suitable for hydrolysis of polyglutamic acid can be used. In some embodiments, the hydrolysis solvent is selected from dioxane, anisole, benzene, chloroform, chlorobenzene, ethyl acetate, nitrobenzene, acetonitrile, dimethylformamide, nitromethane, methanol, acetic acid, acetone, n-butanol, butyl acetate, carbon tetrachloride, cyclohexane, 1,2-dichloroethane, dichloromethane, dimethylsulfoxide, ethanol, diethyl ether, heptane, hexane, methanol, methyl-t-butyl ether, methyl ethyl ketone, pentane, n-propanol, isopropanol, diisopropyl ether, tetrahydrofuran, toluene, trichloroethylene, water, xylene, and any mixture thereof.

Preferred solvents include polar solvents, such as a polar protic or a polar aprotic solvent. The hydrolyzing conditions can be conducted in a solvent selected from among aqueous solvents, alcoholic solvents, or any mixture thereof. Exemplary solvents include, but are not limited to, methanol, ethanol, propanol, butanol, water, or any mixture thereof. Preferred solvents include acetic acid, dichloroacetic acid, and a mixture of acetic acid and dichloroacetic acid.

vii. Measuring the Extent of Hydrolysis

In some embodiments, the hydrolysis of the polyglutamic acid is monitored. For example, a measurement can be taken to monitor the extent of hydrolysis of the starting polyglutamic acid polymer. The measurement can be used to determine whether the target weight average molecular weight of the polyglutamic acid has been produced. The measurement can also be used to determine the weight average molecular weight of the polyglutamic acid contained in the hydrolyzing solution at any selected stage of the process.

Various techniques for monitoring the hydrolysis of the polyglutamic acid can be used. For example, the hydrolysis of the polyglutamic acid can be monitored by techniques including, but not limited to, size exclusion chromatography-high pressure liquid chromatography (SEC-HPLC) using appropriate molecular weight detection technology (e.g., light scattering), small angle neutron scattering (SANS), X-ray scattering, sedimentation velocity, size exclusion chromatography, high performance liquid chromatography, gas chromatography-mass spectrometry (GC/MS), liquid chromatography-mass spectrometry (LC/MS), matrix-assisted laser desorption/ionization mass spectrometry (MALDI-MS), electrospray ionization mass spectrometry (ESI/MS), fast atom bombardment mass spectrometry (FAB-MS), inductively coupled plasma-mass spectrometry (ICP-MS), accelerator mass spectrometry (AMS), thermal ionization-mass spectrometry (TIMS), spark source mass spectrometry (SSMS), osmometry, light scattering, ultracentrifugation, cryoscopy, ebulliometry, end-group analysis, titration, freezing-pint depression, boiling-point elevation, osmotic pressure, or any other method of determining the molecular weight of polymers known in the art. If two or more methods for determining the molecular weight of the polyglutamic acid polymer produce different molecular weight values, then the molecular weight value obtained by SEC-HPLC is preferred.

In some embodiments, a measurement of the entire hydrolyzing solution is taken. In some embodiments, a sample or aliquot of the hydrolyzing solution is measured. In some embodiments, both the entire hydrolyzing solution and a sample of the hydrolyzing solution are measured to determine the extent of hydrolysis of the starting polyglutamic acid.

viii. Multiple Measurements

In some embodiments, multiple measurements are taken to monitor the extent of hydrolysis of the starting polyglutamic acid polymer. In some embodiments, multiple measurements are taken to determine whether the target average molecular weight of the polyglutamic acid has been produced. The number of measurements can be between 2 and 40, or more than 40. Multiple measurements can be taken at various time points. For example, measurements can be at intervals between times points in the range of about 1 minute to about 120 minutes.

ix. Correlating Hydrolysis Time with Polyglutamic Acid Molecular Weight

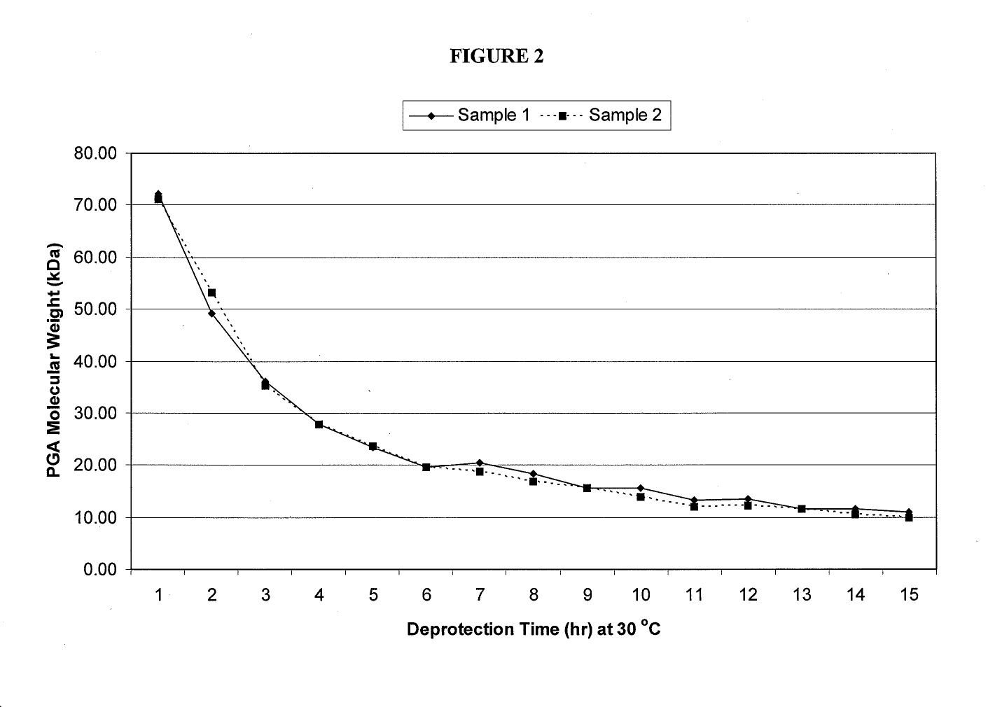

The multiple measurements described above can be used to correlate the amount of time in which the polyglutamic acid is subjected to hydrolyzing conditions and the weight average molecular weight of the polyglutamic acid. For example, plots of the weight average molecular weight of polyglutamic acid versus time are shown in FIGS. 1 and 2. These plots were generated by taking measurements of the weight average molecular weight of polyglutamic acid at various time intervals from a solution containing polyglutamic acid and hydrolyzing reagents. Such plots can be used to determine the amount of time a starting polyglutamic acid polymer should be subjected to hydrolyzing conditions to produce a desired product polyglutamic acid polymer with a selected target weight average molecular weight.

x. Selecting Hydrolyzing Conditions

Based on a correlation of hydrolysis conditions and polyglutamic acid weight average molecular weight, it is possible to select hydrolyzing conditions that are effective to produce a product polyglutamic acid polymer with a target weight average molecular weight.

In some embodiments, the correlation of hydrolysis time and polyglutamic acid weight average molecular weight can be used to select the amount of time that is effective to produce a product polyglutamic acid polymer with a target weight average molecular weight. For example, according to the hydrolyzing conditions utilized to generate FIG. 1, it is possible to select a hydrolysis time of approximately 1 hour to produce a product polyglutamic acid with a weight average molecular weight of approximately 75 kDa. Furthermore, according to FIG. 1, it is possible to select a hydrolysis time of approximately 2 hours to produce a product polyglutamic acid with a weight average molecular weight of approximately 50 kDa. According to FIG. 1, it is possible to select a hydrolysis time of approximately 3 hours to produce a product polyglutamic acid with a weight average molecular weight of approximately 40 kDa. According to FIG. 1, it is possible to select a hydrolysis time of approximately 4-5 hours to produce a product polyglutamic acid with a weight average molecular weight of approximately 34 kDa. According to FIG. 1, it is possible to select a hydrolysis time of approximately 6 hours to produce a product polyglutamic acid with a weight average molecular weight of approximately 26 kDa. According to FIG. 1, it is possible to select a hydrolysis time of approximately 7 hours to produce a product polyglutamic acid with a weight average molecular weight of approximately 20 kDa.

According to the hydrolyzing conditions utilized to generate FIG. 2, it is possible to select a hydrolysis time of approximately 1 hour to produce a product polyglutamic acid with a weight average molecular weight of approximately 72 kDa. Furthermore, according to FIG. 2, it is possible to select a hydrolysis time of approximately 2 hours to produce a product polyglutamic acid with a weight average molecular weight of approximately 50 kDa. According to FIG. 2, it is possible to select a hydrolysis time of approximately 3 hours to produce a product polyglutamic acid with a weight average molecular weight of approximately 36 kDa. According to FIG. 2, it is possible to select a hydrolysis time of approximately 4 hours to produce a product polyglutamic acid with a weight average molecular weight of approximately 28 kDa. According to FIG. 2, it is possible to select a hydrolysis time of approximately 5 hours to produce a product polyglutamic acid with a weight average molecular weight of approximately 23 kDa. According to FIG. 2, it is possible to select a hydrolysis time of approximately 6-8 hours to produce a product polyglutamic acid with a weight average molecular weight of approximately 20 kDa. According to FIG. 2, it is possible to select a hydrolysis time of approximately 9-10 hours to produce a product polyglutamic acid with a weight average molecular weight of approximately 16 kDa. According to FIG. 2, it is possible to select a hydrolysis time of approximately 11-12 hours to produce a product polyglutamic acid with a weight average molecular weight of approximately 13 kDa. According to FIG. 2, it is possible to select a hydrolysis time of approximately 13-15 hours to produce a product polyglutamic acid with a weight average molecular weight of approximately 12 kDa.

Various correlations between hydrolysis conditions and polyglutamic acid weight average molecular weight described herein can be used to produce a polyglutamic acid polymer with a selected target weight average molecular weight.

xi. Purification

Optionally, the product polyglutamic acid may then be isolated and/or purified. Suitable methods known to those skilled in the art can be used to isolate and/or purify the product polyglutamic acid. If needed and/or desired, the product polyglutamic acid may be dried by any suitable method known to those skilled in the art. For example, polyglutamic acid can be precipitated out of solution by adding a reagent. In some embodiments, the reagent can be acetone. Any product polyglutamic acid precipitate that forms can then be filtered and washed, for example with acetone. Optionally, the product polyglutamic acid can be purified by any suitable method. For example, the product polyglutamic acid can be dissolved into a sodium bicarbonate solution, dialyzed in water using a cellulose membrane, and the product polyglutamic acid can be lyophilized and isolated.

As described herein, the product polyglutamic acid obtained from the selected hydrolyzing conditions has a weight average molecular weight less than the starting polyglutamic acid. Methods for determining the weight average molecular weight of the product glutamic acid are described herein. In some embodiments, the weight average molecular weight of the product polyglutamic acid can be in the range of about 35 kDa to about 12 kDa.

As discussed above, in some embodiments, one advantage of the processes described herein is the ability to obtain the product polyglutamic acid with a desired weight average molecular weight within a relatively narrow range of kiloDaltons (kDa). In some embodiments, the product polyglutamic acid can have a weight average molecular weight that is within about ±5 kDa of the selected target second weight average molecular weight. In other embodiments, the product polyglutamic acid can have a weight average molecular weight that is within about ±3 kDa, ±1.0 kDa, ±0.5 kDa, ±0.2 kDa, ±0.1 kDa or ±0.05 kDa of the selected target second weight average molecular weight.

xii. Polydispersity

The hydrolyzing conditions described herein can be used to produce a product polyglutamic acid polymer with a low polydispersity index. In some embodiments, the product polyglutamic acid polymer can have a polydispersity less than 1.5, less than 1.25 or less than 1.1. In some embodiments, the product polyglutamic acid polymer has a polydispersity of about 1.1, 1.2, 1.3, 1.4, 1.5, 1.6, 1.7, 1.8, 1.9, or 2.0. In some embodiments, the product polyglutamic acid polymer has a polydispersity of between 1.01 and 1.09. In some embodiments, the product polyglutamic acid has a polydispersity of about 1.01, 1.02, 1.03, 1.04, 1.05, 1.06, 1.07, 1.08, or 1.09.

### EXAMPLES

The following examples are provided for the purposes of further describing the embodiments described herein, and do not limit the scope of the claims.

### Example 1

### Hydrolysis of Commercially Obtained PGA

Polyglutamic acid (PGA) was obtained from Sigma Aldrich Chemical Co. having a molecular weight of 17 kDa. The commercial PGA was treated using the hydrolyzing conditions shown in Table 1. The weight average molecular weights of the resulting product PGA are also shown in Table 1.

TABLE 1

| | | Hydrolyzing Conditions | | | |
|---|---|---|---|---|---|
| | | | | Time for heating at 50° C. | MW (kDa) |
| | Starting | | | and (sitting at room | determined by |
| Sample | Material | Cl₂CHCO₂H | 33%HBr—AcOH | temperature) | GPC-MALS |
| 1 | Sigma PGA, | — | — | 0 h + (0 h) | 17.00 |
| | 17 kDa, | | | | |
| 2 | Sigma PGA, | 40 ml/g | 3.75 ml/g | 0 h + (3 h) | 15.33 |
| | 17 kDa | | | | |
| 3 | Sigma PGA, | 40 ml/g | 3.75 ml/g | 1 h + (1 h) | 12.85 |
| | 17 kDa | | | | |
| 4 | Sigma PGA, | 40 ml/g | 3.75 ml/g | 2 h + (2 h) | 8.25 |
| | 17 kDa | | | | |
| 5 | Sigma PGA, | 37 ml/g | 6.6 ml/g | 2 h + (4 h) | 6.38 |
| | 17 kDa | | | | |

### Example 2

### Synthesis of PGA-Benzylic Ester

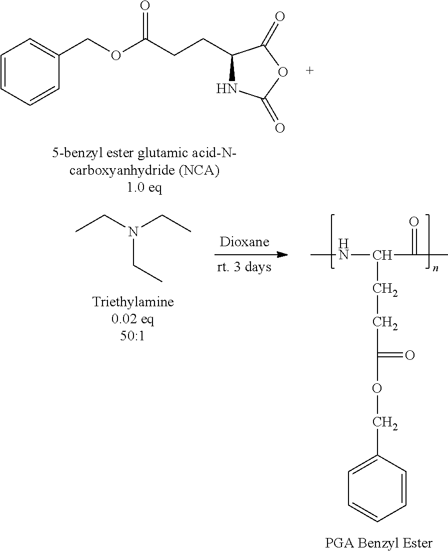

To an oven-dried 500 mL round bottom flask, equipped with a Teflon coated stir bar, was added 10 grams (38 mmol, 1 equiv.) of 5-benzyl ester glutamic acid-N-carboxyanhydride (NCA) and 190 mL of dioxane. The resulting solution was purged with argon for 5 minutes. About 0.106 mL (0.76 mmol, 0.02 equiv.) of freshly distilled triethylamine (0.02 equiv.) was then added. The reaction mixture was purged with argon for an additional 5 minutes, and stirred for 10 minutes. Stirring was then stopped and the reaction mixture was allowed to stand for 72 hours. The reaction mixture was then slowly poured into 1000 mL of rapidly stirring anhydrous ethanol. The product precipitated out as long white, fibrous filaments. The mixture was filtered, and the product was isolated and washed with 250 mL ethanol. Any residual solvent was removed in vacuo. The weight average molecular weight of the resulting PGA sample (used as a starting PGA sample for Example 3) was determined using GPC with light scattering molecular weight detection. Two additional PGA samples were made by similar procedures. Further details regarding the conditions and weight average molecular weights of the resulting PGA samples are set forth in Table 2.

TABLE 2

| | Starting material and | PGA benzyl ester | |
|---|---|---|---|
| | solvent for | Molecular Weight (GPC- | Poly- |
| Sample | polymerization | light scattering detection) | dispersity |
| 1 | Commercial NCA and | 191 kDa | 1.162 |
| | dioxane | | |
| 2 | Recryst. NCA and | 221 kDa | 1.032 |
| | Distilled dioxane | | |
| 3 | Commercial NCA and | 240 kDa | 1.123 |
| | distilled dioxane with Na | | |

### Example 3

### Hydrolysis of PGA

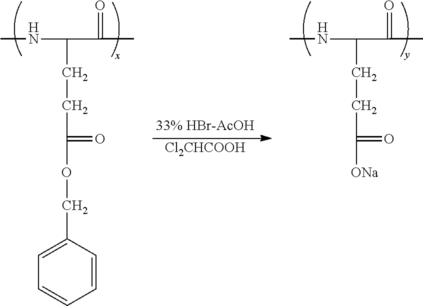

To an oven-dried 100 mL round bottom flask, equipped with a Teflon coated stir bar and gas adaptor, was added 1.0 g (4.57 mmol, 1 equiv.) of PGA-gamma benzyl ester obtained from Example 2 (having a weight average molecular weight molecular weight of 191 kDa) and 40 mL of dichloroacetic acid. The reaction mixture was placed under an atmosphere of argon and stirred for 15 minutes to allow for partial dissolution of the ester. 3.5 mL (28.5 mmol, 6.24 equiv) of the 33% HBr-AcOH solution was added via syringe. The reaction mixture was then stirred for about 6 hours. Acetone (50 mL) was added, and a white precipitate formed. The resulting slurry was filtered and washed with acetone (50 mL) to obtain a solid. The solid was dissolved in 1 N aq. sodium bicarbonate until a pH of ˜8 was reached (approximately 20 mL). The solution was placed into dialysis tubing and dialyzed in 4 L deionized water for about 1 hour. After 1 hour, 100% of the water was changed, and the dialysis was continued for another hour. This process was repeated 2 more times, and then the solution was dialyzed overnight. The dialyzed solution was filtered through a 0.45 μm cellulose acetate membrane and lyophilized to remove the water. The product PGA obtained was a white solid (0.18 g, 31% yield, weight average molecular weight of 16.80 kDaltons).

The procedure of Example 3 was conducted in which aliquots were taken from the reaction mixture at 1, 2, 3, 4, 5, 6 and 7 hours after the addition of 33% HBr-AcOH. The product PGA from the aliquots was purified and isolated using the procedure described in Example 3. The weight average molecular weights of the product PGA from the aliquots were determined and are provided in Table 3.

TABLE 3

| | Sample 1 | Sample 2 | Sample 3 | Sample 4 | Sample 5 | Sample 6 |
|---|---|---|---|---|---|---|
| Time | MW | MW | MW | MW | MW | Avg. MW |
| (hr) | (kDa) | (kDa) | (kDa) | (kDa) | (kDa) | (kDa) |
| 1 | 75.55 | 64.36 | — | 73.63 | 87.91 | 75.36 |
| 2 | 52.35 | 43.69 | 40.40 | 49.34 | 57.73 | 48.70 |
| 3 | 40.25 | 31.21 | — | 39.83 | 48.8 | 40.02 |
| 4 | — | 25.40 | 29.21 | 38.34 | 41.86 | 33.70 |
| 5 | 30.59 | 20.76 | — | 33.25 | 43.5 | 32.03 |
| 6 | 23.46 | 17.77 | 25.72 | 29.31 | 32.23 | 25.70 |
| 7 | 17.96 | 16.88 | — | 26.34 | — | 20.39 |

* MW = weight average molecular weight as determined by GPC-light scattering detection

The plot shown in FIG. 1 was generated using the data in Table 3. One skilled in the art can select a target weight average molecular weight and use this plot to determine the time needed to hydrolyze a starting polyglutamic acid in order to obtain a product polyglutamic acid, wherein the product polyglutamic acid has a weight average molecular weight that is within about ±10 kDa of a selected target second weight average molecular weight.

### Example 4

### Hydrolysis of PGA

A first sample of PGA benzylic ester (5.0 g, 22.85 mmol, 1 equiv) with a starting weight average molecular weight of 130 kDa and dichloroacetic acid (200 mL) was added to an oven-dried 500 mL round bottom flask equipped with a Teflon magnetic stir bar under an argon atmosphere. The flask was lowered into a pre-heated 30° C. oil bath. The resulting suspension was allowed to stir for 15 minutes to allow partial dissolution of the ester. A solution of HBr-AcOH (17.5 mL, 100.1 mmol, 4.37 equiv) was added via syringe. One hour after the addition of the HBr-AcOH solution, a 2.0 mL aliquot of solution was removed via syringe (all the PGA-ester had gone into solution at that time). The aliquot was placed into a centrifuge tube and diluted with 33 mL acetone and vortexed to evenly disperse the polymer in the solvent mixture. The tube was then centrifuged for 5 minutes at 20° C. at 3000 rpm. The polymer formed a tight plug at the bottom of the tube. The supernatant was decanted and an additional 33 mL of acetone was added to the tube. The tube was then vortexed and centrifuged as before. After the resulting supernatant was decanted, the polymer plug was dissolved in 10 mL of 1 N aqueous sodium bicarbonate. Each subsequent hour for an additional 14 hours, a 2.0 mL aliquot of the reaction mixture was removed and worked up as described above. The polymer was characterized by gel permeation chromatography with light scattering detector for weight average molecular weight. The weight average molecular weights of the product PGA were determined.

A second sample of PGA benzylic ester with a starting weight average molecular weight of 130 kDa was hydrolyzed according to the same procedure described above. The weight average molecular weights of the product PGA from the first and second samples are shown in Table 4.

TABLE 4

| Time | Sample 1 | Sample 2 |
|---|---|---|
| (hr) | MW (kDa) | MW (kDa) |
| 1 | 72.14 | 72.14 |
| 2 | 49.21 | 49.21 |
| 3 | 36.15 | 36.15 |
| 4 | 27.78 | 27.78 |
| 5 | 23.44 | 23.44 |
| 6 | 19.71 | 19.71 |
| 7 | 20.37 | 20.37 |
| 8 | 18.4 | 18.4 |
| 9 | 15.61 | 15.61 |
| 10 | 15.7 | 15.7 |
| 11 | 13.34 | 13.34 |
| 12 | 13.59 | 13.59 |
| 13 | 11.53 | 11.53 |
| 14 | 11.67 | 11.67 |
| 15 | 11.08 | 11.08 |

* MW = weight average molecular weight as determined by GPC-light scattering detection

The plot shown in FIG. 2 was generated using the data in Table 4 and shows the weight average molecular weight of the two sample polyglutamic acids under hydrolyzing conditions over a period of several hours. One skilled in the art can select a target weight average molecular weight and use this plot to determine the time needed to hydrolyze a starting polyglutamic acid in order to obtain a product polyglutamic acid, wherein the product polyglutamic acid has a weight average molecular weight that is within about ±10 kDa of a selected target second weight average molecular weight.

### Example 5

### Hydrolysis of PGA

The reproducibility of hydrolyzing conditions was demonstrated by hydrolyzing eight samples of a PGA polymer with a starting weight average molecular Weight of 130 kDa. Briefly, a sample PGA benzylic ester (5.0 g, 22.85 mmol, 1 equiv) and dichloroacetic acid (200 mL) were added to an oven-dried 500 mL round bottom flask equipped with a Teflon magnetic stir bar under an argon atmosphere. The flask was lowered into a pre-heated 30° C. oil bath. The resulting suspension was stirred for 15 minutes to allow partial dissolution of the ester. A solution of HBr-AcOH (17.5 mL, 100.1 mmol, 4.37 equiv) was added. The reaction was stirred for 6 hours. The reaction was poured into 1500 mL of a rapidly stirring mixture of 10% hexane in ethyl acetate. Product precipitated out as a clear gelatinous solid over a period of 15 minutes. The resulting mixture was filtered through a Grade 54 paper filter. The resulting solid was collected and washed with 2×250 mL ethyl acetate. The material was transferred to an Erlenmeyer flask equipped with a stir bar. Then 250 mL of 1 N sodium bicarbonate solution was added to the flask and the material dissolved. The solution was placed into a separation funnel and the aqueous phase (lower layer) was separated from the upper organic layer (benzyl bromide side product and some remaining ethyl acetate). The aqueous layer was placed into a dialysis membrane and dialyzed against 4 L DI water for 1 hour. A 100% water change was performed and then dialyzed for another hour. This process was repeated twice more and then dialyzed overnight. The solution was filtered through a Grade No. 50 filter paper and lyophilized to remove water. The resulting polymer was characterized by ¹H-NMR spectroscopy for composition and gel permeation chromatography with light scattering detector for weight average molecular weight. The weight average molecular weights of the product PGA for each of the eight samples were determined and are provided in Table 5.

TABLE 5

| Sample | Scale | Starting MW | Yield | Product MW |
|---|---|---|---|---|
| 1 | 5 g | 130 kDa | 44% | 20.53 kDa |
| 2 | 5 g | 130 kDa | 61% | 19.34 kDa |
| 3 | 5 g | 130 kDa | 71% | 21.27 kDa |
| 4 | 5 g | 130 kDa | 79% | 19.71 kDa |
| 5 | 5 g | 130 kDa | 74% | 21.21 kDa |
| 6 | 5 g | 130 kDa | 87% | 21.34 kDa |
| 7 | 5 g | 130 kDa | 83% | 18.27 kDa |
| 8 | 5 g | 130 kDa | 94% | 19.56 kDa |

### Example 6

### Hydrolysis of PGA

The versatility of the hydrolyzing conditions described herein was demonstrated by hydrolyzing six samples of a PGA polymer with starting weight average molecular weights of either 130 kDa or 270 kDa, and sample sizes of between 5 and 50 grams. Briefly, a sample PGA benzylic ester and dichloroacetic acid were added to an oven-dried round bottom flask equipped with a Teflon magnetic stir bar under an argon atmosphere. The flask was lowered into a pre-heated 30° C. oil bath. The resulting suspension was stirred for 15 minutes to allow partial dissolution of the ester. A solution of HBr-AcOH (4.37 equiv) was added. The reaction was stirred for 6 hours. The reaction was poured into a rapidly stirring mixture of 10% hexane in ethyl acetate. Product precipitated out as a clear gelatinous solid over a period of 15 minutes. The resulting mixture was filtered through a Grade 54 paper filter. The resulting solid was collected and washed with ethyl acetate twice. The material was transferred to an Erlenmeyer flask equipped with a stir bar. Then a 1 N sodium bicarbonate solution was added to the flask and the material dissolved. The solution was placed into a separation funnel and the aqueous phase (lower layer) was separated from the upper organic layer (benzyl bromide side product and some remaining ethyl acetate). The aqueous layer was placed into a dialysis membrane and dialyzed against DI water for 1 hour. A 100% water change was performed and then dialyzed for another hour. This process was repeated twice more and then dialyzed overnight. The solution was filtered through a Grade No. 50 filter paper and lyophilized to remove water. The resulting polymer was characterized by ¹H-NMR spectroscopy for composition and gel permeation chromatography with light scattering detector for weight average molecular weight. The weight average molecular weights of the product PGA for each of the six samples were determined and are provided in Table 6.

TABLE 6

| Sample | Scale | Starting MW | Yield | Product MW |
|---|---|---|---|---|
| 1 | 5 g | 130 kDa | 44% | 20.53 kDa |
| 2 | 5 g | 130 kDa | 61% | 19.34 kDa |
| 3 | 15 g | 270 kDa | 61% | 19.46 kDa |
| 4 | 20 g | 130 kDa | 51% | 20.09 kDa |
| 5 | 50 g | 270 kDa | 52% | 20.52 kDa |
| 6 | 50 g | 270 kDa | 54% | 19.22 kDa |

It will be understood by those of skill in the art that numerous and various modifications can be made without departing from the spirit of the present application. Therefore, it should be clearly understood that the forms disclosed in the present application are illustrative only and not intended to limit the scope of the claims.

What is claimed is:

1. A process for preparing polyglutamic acid comprising:
obtaining a starting polyglutamic acid having a first weight average molecular weight equal to or greater than 80 kDa;
selecting a target second weight average molecular weight of polyglutamic acid that is less than 80 kDa;
selecting hydrolyzing conditions that are effective to reduce the first weight average molecular weight of the starting polyglutamic acid to the selected target second weight average molecular weight of polyglutamic acid; and
hydrolyzing the starting polyglutamic acid under the selected hydrolyzing conditions to thereby obtain a product polyglutamic acid, wherein the product polyglutamic acid has a weight average molecular weight that is within about ±10 kDa of the selected target second weight average molecular weight.

2. A process for preparing polyglutamic acid comprising:
obtaining a starting polyglutamic acid having a first weight average molecular weight equal to or greater than 185 kDa;
selecting a target second weight average molecular weight of polyglutamic acid that is less than 185 kDa;
selecting hydrolyzing conditions that are effective to reduce the first weight average molecular weight of the starting polyglutamic acid to the selected target second weight average molecular weight of polyglutamic acid; and
hydrolyzing the starting polyglutamic acid under the selected hydrolyzing conditions to thereby obtain a product polyglutamic acid, wherein the product polyglutamic acid has a weight average molecular weight that is within about ±10 kDa of the selected target second weight average molecular weight.

3. The process of claim 1, wherein the product polyglutamic acid has a weight average molecular weight that is within about ±5 kDa of the selected target second weight average molecular weight.

4. The process of claim 1, wherein the product polyglutamic acid has a weight average molecular weight that is within about ±3.0 kDa of the selected target second weight average molecular weight.

5. The process of claim 1, wherein the starting polyglutamic acid has a first weight average molecular weight equal to or greater than 190 kDa.

6. The process of claim 1, wherein the starting polyglutamic acid has a first weight average molecular weight equal to or greater than 220 kDa.

7. The process of claim 1, wherein the starting polyglutamic acid has a first weight average molecular weight equal to or greater than 240 kDa.

8. The process of claim 1, wherein the starting polyglutamic acid has a first weight average molecular weight in the range of 80 kDa to about 300 kDa.

9. The process of claim 1, wherein the starting polyglutamic acid is a reaction product of a glutamic acid ester and an amine.

10. The process of claim 1, wherein the selected target second weight average molecular weight is equal to or less than 40 kDa.

11. The process of claim 1, wherein the selected target second weight average molecular weight is in the range of about 35 kDa to about 15 kDa.

12. The process of claim 1, wherein the starting polyglutamic acid is hydrolyzed under the selected hydrolyzing conditions for at least a total of 2 hours.

13. The process of claim 1, wherein the starting polyglutamic acid is hydrolyzed under the selected hydrolyzing conditions for at least a total of 3 hours.

14. The process of claim 1, wherein the starting polyglutamic acid is hydrolyzed under the selected hydrolyzing conditions for at least a total of 5 hours.

15. The process of claim 1, wherein the starting polyglutamic acid is hydrolyzed under the selected hydrolyzing conditions for less than a total of 8 hours.

16. The process of claim 1, wherein the selected hydrolyzing conditions comprise reacting the starting polyglutamic acid with an acid.

17. The process of claim 16, wherein the acid is HBr-acetic acid.

18. The process of claim 1, wherein the selected hydrolyzing conditions comprise subjecting the starting polyglutamic acid to a first temperature greater than 25° C.

19. The process of claim 18, wherein the selected hydrolyzing conditions further comprises subjecting the starting polyglutamic acid to a second temperature not equal to the first temperature.

20. The process of claim 19, wherein the starting polyglutamic acid polymer is hydrolyzed at the first temperature for a first period of time and the second temperature for a second period of time.
